(19) United States
(12) Patent Application Publication
(10) Pub. No.: US 2020/0405142 A1
(43) Pub. Date: Dec. 31, 2020

(54) MEDICAL USE CAMERA

(71) Applicant: Innovasis, Inc., Salt Lake City, UT (US)

(72) Inventor: Camden Whitaker, Wichita, KS (US)

(21) Appl. No.: 16/916,834

(22) Filed: Jun. 30, 2020

Related U.S. Application Data
(60) Provisional application No. 62/868,963, filed on Jun. 30, 2019.

(51) Int. Cl. A61B 1/317; A61B 1/05; A61B 1/00; A61B 1/045; A61B 1/008

(52) U.S. Cl. CPC A61B 1/317; A61B 1/05; A61B 1/00066; A61B 1/045; A61B 1/00032; A61B 1/008; A61B 1/00096; A61B 1/00039; A61B 1/00154; A61B 1/00045; A61B 1/00016

(57) ABSTRACT

A video and still frame camera for medical use is described. In some embodiments, the camera is sized to be used in orthopedic surgery. The camera comprises a lens that converts the light signals into electrical signals, processing components for the electrical signal, and a wireless transmitter for transmitting the camera images.

### PRIORITY INFORMATION

This application claims priority of U.S. Provisional Application Ser. No. 62,868,963, filed on Jun. 30, 2019, the contents of which are incorporated herein by reference.

### TECHNICAL FIELD

The present disclosure relates generally to video cameras for medical use, more specifically, to a system used in the spinal area.

### BACKGROUND

For the practitioner, the field of diagnostic imaging, such as endoscopy, has allowed for the viewing of objects, internal mechanisms and the like with minimal disruption to the patient. These imaging tools have been used in a wide variety of settings for detailed inspection. Many of these imaging system components must utilize a cabling means to function. These cables transfer electricity, optical information and mechanical means but may physically interfere with objects and persons in the room.

### BRIEF DESCRIPTION OF THE DRAWINGS

FIG. 1 is a perspective view of a medical use camera in accordance with one or more embodiments of the present disclosure.

FIG. 2 is a side view of a medical use camera in accordance with one or more embodiments of the present disclosure.

FIG. 3 is a perspective view of a medical use camera in accordance with one or more embodiments of the present disclosure.

FIG. 4 is a perspective view of a medical use camera in accordance with one or more embodiments of the present disclosure.

FIG. 5 is a perspective view of a medical use camera in accordance with one or more embodiments of the present disclosure.

FIG. 6 is a block diagram of a medical use camera in accordance with one or more embodiments of the present disclosure.

### DETAILED DESCRIPTION

Embodiments of the present disclosure include a small, easily manipulated camera device that can be used in a sterile environment, such as orthopedics. The camera can be sized for easy manipulation by a user and the shutter can be positioned and dimensioned such that the camera can be used both for open and minimally invasive surgery.

In some embodiments, the camera has a shutter on the distal end and a handle at the proximal end. The shutter protects the camera lens and can be opened by the user when needed. In embodiments wherein the camera has a shutter, a switch may be provided to open the shutter, or it may be opened automatically

In some embodiments, the handle is ergonomically designed for ease of handling and use. In some embodiments, a zoom button and/or focus button are provided for the user. In some embodiments, the camera auto focuses itself.

In some embodiments, the camera includes a LED or other light source so that the target area will be visible to the viewer.

The camera assembly can include a battery to power the camera, a shutter, a lens, focus mechanisms, light source, data storage and/or data transmission module. WiFi, BlueTooth, or other wireless communication protocols can be used to wirelessly connect the camera to a viewing source such as a screen. The camera may also be wirelessly connected to a data recording device or may contain such a device.

Before describing various embodiments of the present disclosure in detail, it is to be understood that this disclosure is not limited to the parameters of the particularly exemplified implants, methods, systems and/or products, which may, of course, vary. Thus, while certain embodiments of the present disclosure will be described in detail, with reference to specific configurations, parameters, features (e.g., components, members, elements, parts, and/or portions), etc., the descriptions are illustrative and are not to be construed as limiting the scope of the claimed invention. In addition, the terminology used herein is for the purpose of describing the embodiments and is not necessarily intended to limit the scope of the claimed invention.

Unless defined otherwise, all technical and scientific terms used herein have the same meaning as commonly understood by one of ordinary skill in the art to which the present disclosure pertains.

Various aspects of the present disclosure, including implants, systems, processes, and/or products may be illustrated with reference to one or more embodiments or implementations, which are exemplary in nature. As used herein, the terms “embodiment” and “implementation” mean “serving as an example, instance, or illustration,” and should not necessarily be construed as preferred or advantageous over other aspects disclosed herein. In addition, reference to an “implementation” of the present disclosure or invention includes a specific reference to one or more embodiments thereof, and vice versa, and is intended to provide illustrative examples without limiting the scope of the invention, which is indicated by the appended claims rather than by the following description

As used throughout this application the words “can” and “may” are used in a permissive sense (i.e., meaning having the potential to), rather than the mandatory sense (i.e., meaning must). Additionally, the terms “including,” “having,” “involving,” “containing,” “characterized by,” as well as variants thereof (e.g., “includes,” “has,” and “involves,” “contains,” etc.), and similar terms as used herein, including the claims, shall be inclusive and/or open-ended, shall have the same meaning as the word “comprising” and variants thereof (e.g., “comprise” and “comprises”), and do not exclude additional, un-recited elements or method steps, illustratively.

It will be noted that, as used in this specification and the appended claims, the singular forms “a,” “an” and “the” include plural referents unless the context clearly dictates otherwise. Thus, for example, reference to a “locking screw” includes one, two, or more locking screws.

As used herein, directional terms, such as “top,” “bottom,” “left,” “right,” “up,” “down,” “upper,” “lower,” “proximal,” “distal,” “vertical,” “horizontal” and the like are used herein solely to indicate relative directions and are not otherwise intended to limit the scope of the disclosure and/or claimed invention.

Various aspects of the present disclosure can be illustrated by describing components that are bound, coupled, attached, connected, and/or joined together. As used herein, the terms “bound,” “coupled”, “attached”, “connected,” and/or “joined” are used to indicate either a direct association between two components or, where appropriate, an indirect association with one another through intervening or intermediate components. In contrast, when a component is referred to as being “directly bound,” “directly coupled”, “directly attached”, “directly connected,” and/or “directly joined” to another component, no intervening elements are present or contemplated. Furthermore, binding, coupling, attaching, connecting, and/or joining can comprise mechanical and/or chemical association.

To facilitate understanding, like reference numerals (i.e., like numbering of components and/or elements) have been used, where possible, to designate like elements common to the figures. Specifically, in the example embodiments illustrated in the figures, like structures, or structures with like functions, will be provided with similar reference designations, where possible.

Specific language will be used herein to describe the example embodiments. Nevertheless, it will be understood that no limitation of the scope of the disclosure is thereby intended. Rather, it is to be understood that the language used to describe the example embodiments is illustrative only and is not to be construed as limiting the scope of the disclosure (unless such language is expressly described herein as essential). Furthermore, multiple instances of an element and or sub-elements of a parent element may each include separate letters appended to the element number. An element label with an appended letter can be used to designate an alternative design, structure, function, implementation, and/or embodiment of an element or feature without an appended letter. Likewise, an element label with an appended letter can be used to indicate a sub-element of a parent element. However, element labels including an appended letter are not meant to be limited to the specific and/or particular embodiment(s) in which they are illustrated. In other words, reference to a specific feature in relation to one embodiment should not be construed as being limited to applications only within said embodiment.

Furthermore, multiple instances of the same element may each include separate letters appended to the element number. For example, two instances of a particular element “20” may be labeled as “20a” and “20b”. In that case, the element label may be used without an appended letter (e.g., “20”) to generally refer to every instance of the element; while the element label will include an appended letter (e.g., “20a”) to refer to a specific instance of the element.

It will also be appreciated that where multiple possibilities of values or a range a values (e.g., less than, greater than, at least, or up to a certain value, or between two recited values) is disclosed or recited, any specific value or range of values falling within the disclosed range of values is likewise disclosed and contemplated herein.

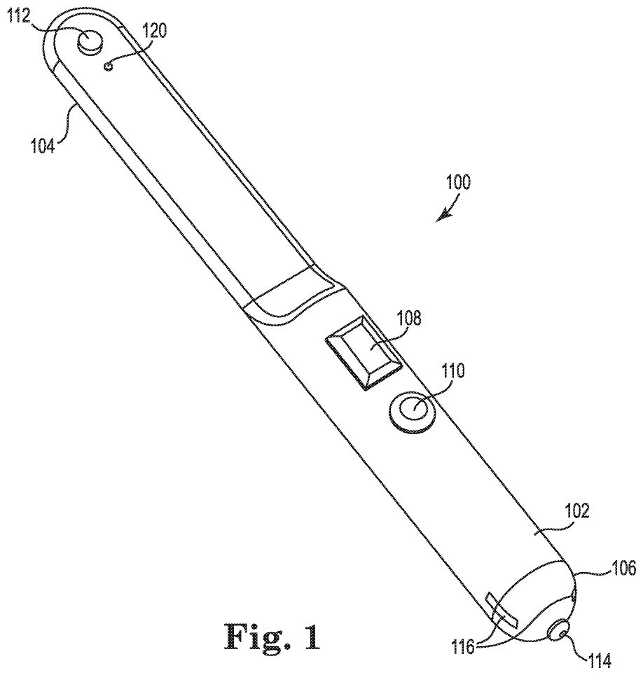

FIG. 1 is a perspective view of a medical use camera 100 in accordance with one or more embodiments of the present disclosure. Camera 100 can be contained in a housing 102. Housing 102 includes a proximal end 106 and a distal end 104. Near distal end 104 can be a shutter 112 which covers the camera lens (not shown). In some embodiments, proximal end 106 is designed to be held by the user when camera 100 is in use. Located near the handle area is zoom feature 108 and auto focus 110. In some embodiments, 110 is also a stop. The camera unit also has an on/off button 114 and may have one or more antennas 116 for transmitting data. While the antennas 116 are shown in FIG. 1 on the outside of housing 102, they can also be located within the housing 102. This embodiment also includes on/off button 114. Also shown is optional light source 120. In some embodiments, a light source such as an LED is used to illuminate the field of view for the camera 100.

Housing 102 can be formed by molding or other process and made of plastic, metal, or any suitable material. In some embodiments, the housing is formed in two parts and can be snapped or fastened together. As shown in FIG. 1, the camera can have a lateral view as the shutter 112 and lens are positioned on a side of the device. While the housing can have any shape, FIG. 1 illustrates a flat distal end 104. The flat surface indicates to the user the direction that the camera is facing. In this embodiment, the zoom 108 and focus button 110 are located on the top of proximal end 106.

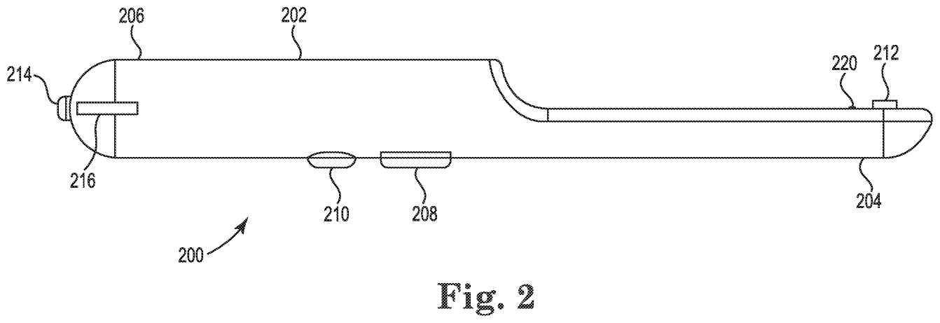

FIG. 2 is a side view of a medical use camera 200 in accordance with one or more embodiments of the present disclosure. The camera 200 shown in FIG. 2 includes some features similar to the camera described with respect to FIG. 1. These features include camera body 202, proximal end 206, distal end 204, zoom button 208, focus button 210, power button 214, antenna 216 (which may also reside inside housing 202), optional light source 220, and shutter/lens 212.

The example illustrated in FIG. 2 differs from that shown in FIG. 1 in that the zoom and focus buttons are on the bottom of the housing 202 instead of on the top. Some users may prefer this configuration.

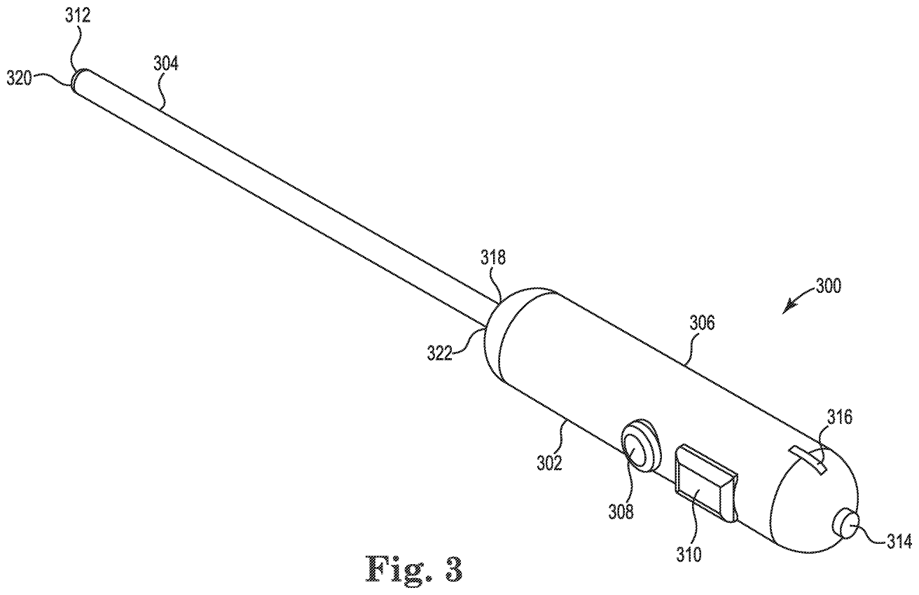

FIG. 3 is a perspective view of a medical use camera 300 in accordance with one or more embodiments of the present disclosure. The camera 300 shown in FIG. 3 has some features similar to the camera described with respect to FIG. 1. These features include camera body 302, proximal end 306, distal end 304, zoom button 308, focus button 310, power button 314, antenna 316 (which may also reside inside housing 302), optional light source 320, and shutter/lens 312.

The example illustrated in FIG. 3 differs from the prior examples in that the distal end 301 is a long cylinder (rod-shaped) with the shutter 312 and lens (not shown) mounted on the distal face. If used, light source 320 would also be mounted on the distal face or near the distal end so that the light is emitted in a distal direction. In this embodiment, distal section 304 may be connected to optional swivel or articulation joint 318 located on the distal end of proximal section 306. This joint 318 can allow the distal section 304 to move away from the longitudinal axis of the proximal section of camera device 300. Also shown is optional connection port 322. As will be discussed below, this would allow proximal section 306, which contains many of the electronics associated with camera 300 to be sterilizable while the distal section 304 which may be in contact with a patient's body to be disposable. The rod-shaped distal end 304 can be used in minimally invasive surgeries which use an access port (hollow tube) as a working channel for surgical instruments.

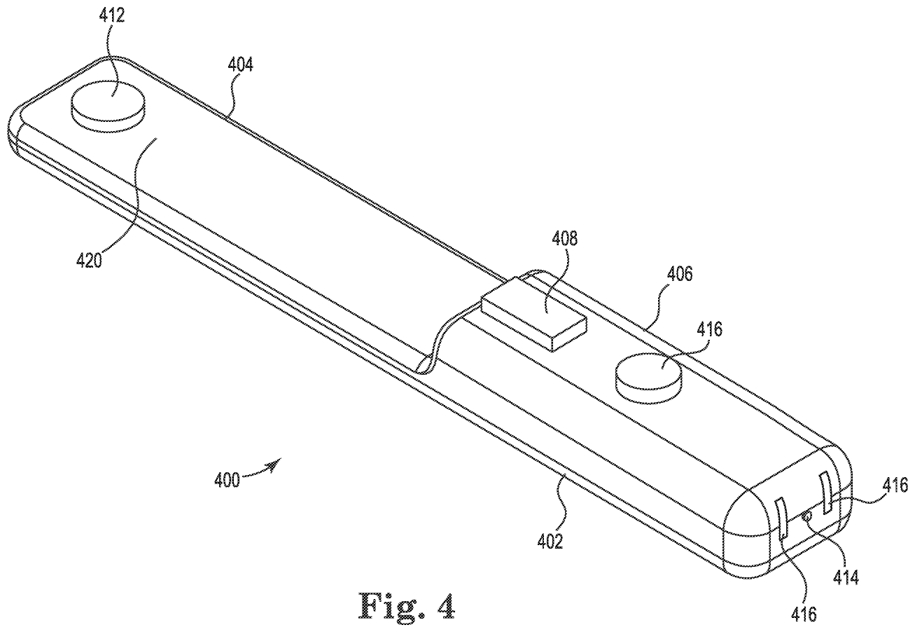

FIG. 4 is a perspective view of a medical use camera 400 in accordance with one or more embodiments of the present disclosure. The camera 400 shown in FIG. 4 has some features similar to the camera described with respect to FIG. 1. These features include camera body 402, proximal end 406, distal end 404, zoom button 408, focus button 410, power button 414, antenna 416 (which may also reside inside housing 402), optional light source 420, and shutter/lens 412.

Camera 400 is similar in design to camera 100 shown in FIG. 1 in that the distal section 404 is flat, but whereas the bottom of camera 100 is slightly rounded, the bottom of 400 is flat as well. This results in a generally rectangular cross section for both proximal section 406 and distal section 404. This allows the camera 400 to have a smaller height which can allow the user to see into spaces that have thin openings.

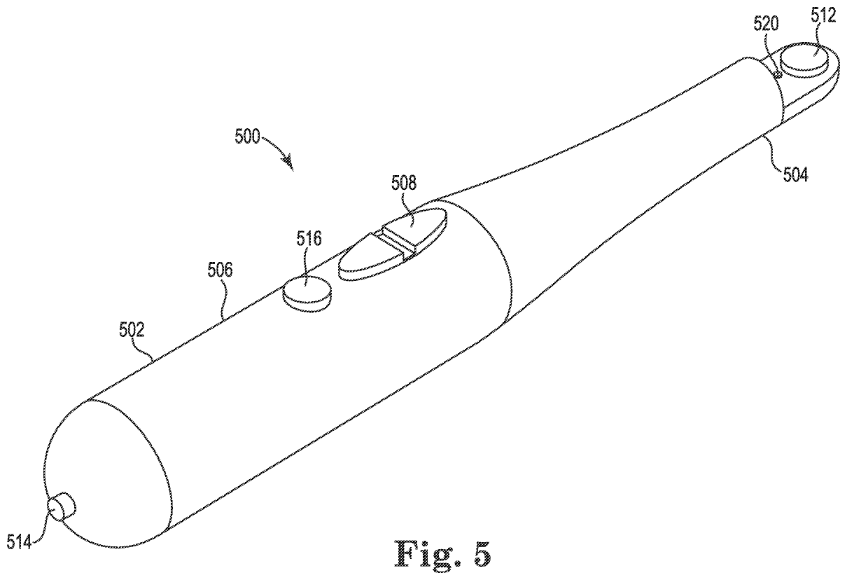

FIG. 5 is a perspective view of a medical use camera 500 in accordance with one or more embodiments of the present disclosure. The camera 500 shown in FIG. 5 has some features similar to the camera described with respect to FIG. 1. These features include camera body 502, proximal end 506, distal end 504, zoom button 508, focus button 510, power button 514, antenna 516 (which may also reside inside housing 502), optional light source 520, and shutter/lens 512. As can be seen in FIG. 5, camera 500 is generally cylindrical tapering to a smaller diameter at distal end 504. Camera 500 has a small notch cut out of the cylinder for shutter/lens 512 and optional light source 520. This version has a lateral camera view.

The zoom and focus features of the described cameras are similar. The zoom provides for drastic changes in the focus point of the camera whereas the focus button provided fine adjustments.

The shutter is optional. In embodiments where the camera does have the shutter, it may have a switch to open and close the shutter. Alternatively, the shutter may open automatically whenever the video is started and then closed again when the video stops. Similarly, it may open automatically whenever a still frame picture is taken.

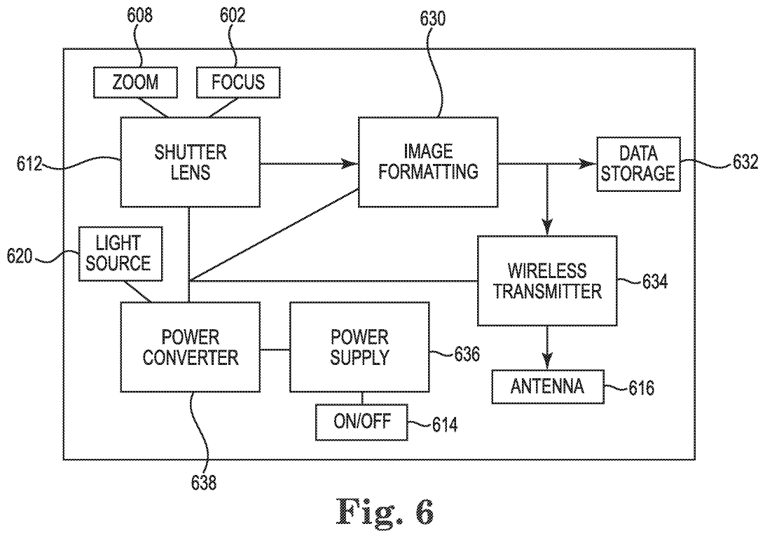

FIG. 6 is a block diagram of a medical use camera in accordance with one or more embodiments of the present disclosure. The cameras described herein have a lens and optional shutter in component 612. The shutter is an openable cover that protects the lens prior/after use and during delivery to the site in the body. In some embodiments, the shutter is not needed. The lens is an image sensor, which converts a pattern of light making up an image into an electrical signal. In one embodiment, the image sensor has a high sensitivity to allow the camera to perform in low light conditions. In one embodiment, the image sensor can have a high dynamic range to allow capturing of gradations on an image from the lightest to the darkest.

The electrical signal from component 612 is sent to the image formatting component 630. Included in component 630 can be a video compression unit, channel encoder, and formatting unit. The video compression unit is the electronic circuit or software that compresses the output of the image sensor. This allows a greater amount of data to be provided in a specified period of time. Once the video output of the image sensor is compressed, the channel encoder detects and corrects errors in the signal improving the fidelity of the video signal. Once encoded, the video image is sent to a formatting unit. Here, the data undergoes the final preparation for being wirelessly transmitted. The preparation will depend upon the type of wireless transmitter that is being used.

From the image formatting, in one embodiment, the video is sent to data storage unit 632 located in the camera body. This data can be accessed via a USB, 9 pin, 15 pin, or similar connection.

From the image formatting, the video is sent to wireless transmitter 634. Through antenna 616, the video image is transmitted. The transmitter 634 can use ultrawideband, Bluetooth, WiFi, or other type of wireless communication. The display for the video feed will have a de-formatting unit in order to display the images. The display can be positioned so that the surgeon who is performing the surgery can see the images.

To power the camera's activities, power supply 636 can be any type of portable energy source, such as for example a nickel metal hydride or lithium rechargeable battery or a disposable alkaline battery. Power converter provides the required power to the optional light source 620, the shutter and lens 612, the image formatting unit 630, and the wireless transmitter 634.

The camera examples described herein have been described in terms of a video camera. In all embodiments, the camera can also or alternatively be a still image camera.

As cameras 100, 200, 300, 400, 500 can be used in a surgical setting, the product may be sterile. In one embodiment, a disposable sleeve will be used. The sleeve, which is sterile, can cover the camera while in use. When the procedure is complete, the sleeve can be discarded. In some embodiments, the disclosed cameras can be low cost and single use. These cameras can be delivered to the surgical setting in a sterile package and disposed of after use. As discussed in relation to camera 300, one part of the camera can be reusable, and another part can be disposable.

In some embodiments, the camera can also include a receiver and processing unit. This would allow the user to communicate with the camera so that patient information, such as an ID number, can be included with the data that is transmitted.

While the disclosed cameras can be used in any part of the body, they may be especially suitable for various orthopedic procedures such as laminectomy, discectomy, spinal fusion, vertebroplasty, and kyphoplasty. The cameras can be useful in open surgical procedures, minimally invasive procedures, and percutaneous orthopedic procedures.

While various features have been described with the word button, any suitable device such as a slide, rotational knob, toggle switch, electronic pad, cradle switch, rocking switch, etc. could be used.

In various embodiments, a focus button was disclosed that allowed the user to control the focusing of the camera lens. In all embodiments, the camera may instead contain an auto focus unit that automatically focuses the lens on whatever image is captured by the lens. In some embodiments, the focus button can be multifunctional. For example, it can be a three way switch wherein moving in one direction (for example distally) will cause the focal point to move further from the lens, moving the button in the other direction (for example proximally) will cause the focal point to move closer to the lens, and pushing the button down will stop and start the video or take a still frame picture. Where the camera has an auto focus feature, the button can be used to stop and start the video or to take still frame pictures. In some embodiments, the camera will not have a zoom feature or button. Instead, the camera will have a single focus length and will rely on either a focus button or an autofocus feature to provide clear images.

The present invention may be embodied in other specific forms without departing from its spirit or essential characteristics. For example, the components, location, and orientation of the features can be modified as needed. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope

What is claimed is:

1. A camera for use in orthopedic procedures comprising:
a body with a proximal end and a distal end;
a camera lens positioned near the distal end of the body that generates an electrical signal;
the proximal end of the body being configured as a handle for the user;
controls to operate the camera, including a power switch to activate the camera;
a signal processing system that converts the electrical signal of the camera lens to a wireless signal and wirelessly transmits the signal; and
a power supply, located in the camera body, to provide power to the camera;
wherein the camera is configured to be of a size and shape that allows the camera to be used in orthopedic procedures.

2. The camera of claim 1, wherein the camera captures and transmits video data.

3. The camera of claim 1, wherein the camera captures and transmits still images.

4. The camera of claim 1, further comprising a focus button for changing the focus location of the lens.

5. The camera of claim 1, further comprising an automatic focus device that automatically focuses on the image that is captured by the lens.

6. The camera of claim 1, further comprising a shutter that covers the lens and a switch for opening and closing the shutter.

7. The camera of claim 4, wherein the focus button acts to stop and start the video.

8. The camera of claim 4, wherein the focus button acts to take still images.

9. The camera of claim 1, further comprising an antenna.

10. The camera of claim 9, wherein the antenna is located on the camera body.

11. The camera of claim 9, wherein the antenna is located within the camera body.

12. The camera of claim 1, further comprising a sterile sleeve that does not interfere with the camera.

13. The camera of claim 1, wherein the distal end of the body is connected to the proximal end of the body with a joint that allows the distal end of the body to move away from the longitudinal axis of the proximal end of the body.

14. The camera of claim 1, wherein the distal end of the camera can be disconnected from the proximal end of the camera.

15. A method for capturing images of an orthopedic surgical site comprising:
performing at least part of an orthopedic procedure at a surgical site;
inserting the distal end of a camera into the surgical site, the camera comprising;
a body with a proximal end and a distal end;
a camera lens positioned near the distal end of the body that captures a light signal and converts it to an electrical signal;
a signal processing system that converts the electrical signal of the camera lens to a wireless signal and wirelessly transmits the wireless signal; and
a power supply, located in the camera body, to provide power to the camera; and
activating the camera to capture still or video images.

16. The method of claim 15, wherein the surgical procedure is a spinal fusion procedure.

17. The method of claim 15, further comprising capturing the wireless signal and converting the wireless signal to a video image.

18. The method of claim 17, further comprising displaying the video image.

19. The method of claim 15, further comprising an articulation joint between the proximal and distal ends of the camera.

20. The method of claim 15, wherein the performing step is being done through a working channel in a minimally invasive fashion and the distal end of the camera is configured to fit down the working channel.
